(19) United States
(12) Patent Application Publication
(10) Pub. No.: US 2025/0248655 A1
(43) Pub. Date: Aug. 7, 2025

(54) SENSOR HOUSING FOR IMPROVED ACCURACY AND ELECTRICAL RELIABILITY

(71) Applicant: KONINKLIJKE PHILIPS N.V., EINDHOVEN (NL)

(72) Inventor: Mathijs DE WIT, ASSENDELFT (NL)

(21) Appl. No.: 18/856,092

(22) PCT Filed: Apr. 11, 2023
(86) PCT No.: PCT/EP2023/059376

§ 371 Date: Oct. 11, 2024

Related U.S. Application Data
(60) Provisional application No. 63/330,380, filed on Apr. 13, 2022.

(51) Int. Cl. A61B 5/00

(52) U.S. Cl. CPC A61B 5/6851; A61B 2560/0462; A61B 2562/168; A61B 2562/227

(57) ABSTRACT

An intraluminal sensing device is provided, which includes a guidewire to be positioned within a body lumen. The guidewire includes a core wire, a sensor configured to obtain medical data from the body lumen, and a sensor housing fixedly attached to the core wire. The sensor housing is a single component that defines an outer profile of the guidewire. The sensor housing includes a core wire lumen configured to receive the core wire through the entire length of the sensor housing such that a cross section of the core wire lumen completely surrounds a cross section of the core wire. The sensor housing also includes an open sensor cavity (different from the core wire lumen) to receive the sensor. The sensor cavity includes an attachment platform fixedly attached to a fixed portion of the sensor, such that a cantilevered portion of the sensor is suspended within the sensor cavity.

### TECHNICAL FIELD

The subject matter described herein relates to intraluminal physiology sensing devices. For example, intravascular catheter or guidewires can include a sensor housing, and associated systems and methods.

### BACKGROUND

Intraluminal physiology sensing devices may be introduced into a body lumen of a patient, and may for example include physiological sensors at a distal end of a catheter or guidewire. Electrical wires may be employed to couple sensing elements at the distal end of the catheter or guidewire with a connector at a proximal end of the catheter or guidewire. Fine-gauge electrical wires may be referred to as filars or conductors. Small-diameter medical devices such as intraluminal (e.g., intravascular) catheters and guidewires may incorporate sensors (e.g., pressure, temperature, flow, or imaging sensors) whose power and communications occur through a multi-filar (e.g., bifilar, trifilar, etc.) electrical conductor bundle. Decreases in device size drive challenges in the device manufacturing process related to securing the sensor to the distal end of the catheter or guidewire, and connecting the conductive filars to it.

For example, recent guidewire devices may in some cases have diameters of 360 microns or smaller. Current construction of such devices may require microcables/filars to be hand soldered/bonded to the sensor, or hand-positioned and then bonded. Additionally, the subassembly consisting of the sensor and filars may be quite delicate. Even under high magnification, manual assembly of these assemblies may be extremely challenging, resulting in high scrap rates and thus high manufacturing costs.

Currently, multiple components may be needed to support and protect the sensor, such as a sensor mount that the sensor is mounted on and a sensor housing surrounding the sensor mount and the sensor.

The mounting method of the sensor and design of the casing around the sensor can impact the performance of the pressure-sensing guidewire, with potential for an inaccurate pressure signal, drift and/or disruption of the electrical signal through: (1) Incomplete protection of the diaphragm allowing contact between the vessel wall and the diaphragm, (2) Mechanical stresses from the components in direct contact with the sensor and the wire body being transmitted to the sensor, (3) Inability of the fluid to reach the sensor diaphragm, (4) Formation of air Bubbles around the sensor diaphragm area, (5) Interruption of electrical contact due to the forces exercised on the sensor during use, or (6) Chemical reactions between different components close to sensor when exposed to the use or storage environment. Additionally, the casing can increase friction between the guidewire and the internal anatomy or covering catheter, which can reduce torqueability, trackability, and/or pushability of the guidewire during medical procedures.

The information included in this Background section of the specification, including any references cited herein and any description or discussion thereof, is included for technical reference purposes only and is not to be regarded as subject matter by which the scope of the disclosure is to be bound.

### SUMMARY

Disclosed are intraluminal physiology sensing devices that include a sensor housing. The present disclosure provides a design that may reduce the potential for inaccurate pressure signals, drift, and disruption of the electrical signal, while also providing for reduced manufacturing defects and reduced complexity of the intraluminal device manufacturing process.

The problems discussed above are addressed by the present disclosure through a rigid housing or casing (e.g., not a flexible tube) generally comprising one or more materials that can, for example, be laid down in layers to produce a 3D structure that isolates the sensor from stresses in the core wire, shaping ribbon, and/or other elements in the distal portion of the guidewire. In an example, the case or housing may be shaped approximately as a rounded rectangular prism, with 5 closed sides and one (partially) open side to expose the diaphragm. Alternatively, the case or housing may be approximately cylindrical and may comprise an approximately rectangular opening to accommodate the sensor. The case or housing may further include an integrated elevated platform to support the proximal side of the sensor, creating a cantilever.

One general aspect includes an intraluminal sensing device, which includes a guidewire configured to be positioned within a body lumen of a patient. The guidewire includes: a core wire; a sensor configured to obtain medical data associated with the body lumen, and a sensor housing fixedly attached to the core wire, where the sensor housing is a single component and defines an outer profile of the guidewire. The sensor housing includes: a core wire lumen configured to receive the core wire through an entire length of the sensor housing such that a cross section of the core wire lumen completely surrounds a cross section of the core wire; and an open sensor cavity different from the core wire lumen and configured to receive the sensor, where the sensor cavity includes an attachment platform fixedly attached to a fixed portion of the sensor such that a cantilevered portion of the sensor is suspended within the sensor cavity. Other embodiments of this aspect include corresponding computer systems, apparatus, and computer programs recorded on one or more computer storage devices, each configured to perform the actions described herein.

Implementations may include one or more of the following features. In some embodiments, the guidewire further includes a pillow disposed beneath the cantilevered portion of the sensor. In some embodiments, the pillow includes trapped air. In some embodiments, the sensor housing further includes an adhesive flow channel or an adhesive flow recess configured to receive an excess of an adhesive, where the attachment platform is fixedly attached to the fixed portion of the sensor by the adhesive. In some embodiments, the sensor housing further includes a fluid flow channel configured to permit access to a sensing element of the sensor by a fluid within the body lumen of the patient. In some embodiments, the sensor housing further includes at least one core wire solder hole configured to receive solder or adhesive, such that the core wire is fixedly attached to the sensor housing by the solder or adhesive. In some embodiments, the guidewire further includes a shaping ribbon extending distally from the sensor housing into a flexible tip coil, where the flexible tip coil is fixedly attached to the sensor housing at a distal end of the sensor housing. In some embodiments, the sensor housing further includes: a channel for receiving the shaping ribbon; or at least one shaping ribbon attachment hole configured to receive solder or adhesive, such that the shaping ribbon is fixedly attached to the sensor housing by the solder or adhesive.

In some embodiments, the guidewire further includes at least one conductor in electrical communication with the sensor, where the sensor housing further includes a conductor support configured to reduce a transference of mechanical stress from the at least one conductor to the sensor. In some embodiments, the at least one conductor includes at least two conductors, and where the conductor support further includes a separation tine for separating two conductors of the at least two conductors. In some embodiments, the sensor cavity is configured to receive the sensor such that a sensing element of the sensor, positioned on the cantilevered portion of the sensor, is facing radially inward toward the core wire, such that a gap exists between the sensing element and a bottom surface of the sensor cavity. In some embodiments, the sensor cavity is configured to receive the sensor such that a sensing element of the sensor is facing radially outward, away from the core wire. In some embodiments, the sensor housing further includes a bridge element extending laterally across the sensor cavity, such that a bottom surface of the bridge element is not in contact with a bottom surface of the sensor cavity. In some embodiments, the bottom surface of the bridge element is configured to apply a downward pressure to a top surface of the fixed portion of the sensor. In some embodiments, the bridge element is configured to provide a space between the bottom surface of the bridge element and a top surface of the cantilevered portion of the sensor.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the sensor housing, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a diagrammatic side view of an intravascular sensing system that includes an intravascular device comprising conductive members and conductive ribbons, according to aspects of the present disclosure.

FIG. 2 is a diagrammatic, perspective view of at least a portion of an example sensor housing, in accordance with at least one embodiment of the present disclosure.

FIG. 3 is a diagrammatic, proximal end view of at least a portion of an example sensor housing, in accordance with at least one embodiment of the present disclosure.

FIG. 4A is a diagrammatic, proximal end view of at least a portion of an example sensor housing, in accordance with at least one embodiment of the present disclosure.

FIG. 4B is a diagrammatic, proximal end view of at least a portion of an example sensor housing, in accordance with at least one embodiment of the present disclosure.

FIG. 5 is a diagrammatic, perspective view of at least a portion of an example sensor housing, in accordance with at least one embodiment of the present disclosure.

FIG. 6 is a diagrammatic, perspective view of at least a portion of an example sensor housing that incorporates fluid flow channels, in accordance with at least one embodiment of the present disclosure.

FIG. 7 is a diagrammatic, perspective view of at least a portion of an example sensor housing that incorporates fluid flow channels, in accordance with at least one embodiment of the present disclosure.

FIG. 8 is a diagrammatic, perspective view of at least a portion of an example sensor housing, in accordance with at least one embodiment of the present disclosure.

FIG. 9 is a diagrammatic, perspective view of at least a portion of an example sensor housing, in accordance with at least one embodiment of the present disclosure.

FIG. 10 is a diagrammatic, cross-sectional view of at least a portion of an example sensor housing, in accordance with at least one embodiment of the present disclosure.

FIG. 11 is a diagrammatic, perspective view of at least a portion of an example sensor housing, in accordance with at least one embodiment of the present disclosure.

FIG. 12 is a diagrammatic, perspective view of at least a portion of an example sensor housing, in accordance with at least one embodiment of the present disclosure.

FIG. 13 is a diagrammatic, perspective view of at least a portion of an example sensor housing, in accordance with at least one embodiment of the present disclosure.

FIG. 14 is a diagrammatic, perspective view of at least a portion of an example sensor housing, in accordance with at least one embodiment of the present disclosure.

FIG. 15 is a diagrammatic, perspective view of at least a portion of an example sensor housing, in accordance with at least one embodiment of the present disclosure.

FIG. 16 is a schematic diagram of a processor circuit, according to at least one embodiment of the present disclosure.

### DETAILED DESCRIPTION

Disclosed are intraluminal physiology sensing devices that include a sensor housing. The present disclosure provides a design that may reduce the potential for inaccurate pressure signals, drift, and disruption of the electrical signal, while also providing for reduced manufacturing defects and reduced complexity of the intraluminal device manufacturing process.

The mounting method of the sensor and design of the casing or housing around the sensor can impact the performance of the pressure-sensing guidewire. Current pressure sensors (e.g., piezoresistive sensors) of pressure-sensing guidewires may for example be mounted within a tubular housing with an opening to provide exposure to blood flow, where the housing is connected to the core of the guidewire. The pressure sensor may be fixed within the casing using adhesive, and mounted such that the sensing element (e.g., a diaphragm located near the distal end of sensor) is cantilevered, as described in U.S. Pat. No. 6,167,763, hereby incorporated by reference as though fully set forth herein. Sensor mount designs and manufacturing/assembly methods can affect the performance of the sensor. Additionally, protruding parts, corners or irregular surfaces of the casing can create friction between the guidewire and internal anatomy, which can reduce torqueability, trackability and pushability. Furthermore, in current pressure-sensing guidewire designs, multiple materials are used to form a casing for the sensor. Multiple materials and/or production aid residues can react with each other, or with the environment, creating contamination which is potentially a biohazard and/or can create stress on the sensor. In current pressure guide wires (e.g., those using piezoresistive technology), the sensor is cantilevered, with the distal portion of the sensor hanging freely, creating an air cavity underneath the distal side of the sensor. Air bubbles can escape from underneath the sensor or out of the distal coil and get in touch with the diaphragm disrupting the pressure measurement. Thus, insufficient access of fluid to the diaphragm can result in and inaccurate pressure signal.

In addition, the sensor is typically fixed in its casing using adhesive. The location of the mounting adhesive can create mechanical stresses, potentially leading to drift in the sensor readings. Stresses from parts surrounding the sensing element can be transferred to the sensing element, potentially affecting sensor readings. Also, in some circumstances, the sensing element can protrude beyond the outer diameter of the guidewire, and/or the pliability of the vessel wall can create direct contact between the vessel wall and the sensing element, creating stresses and affecting readings. Stresses from the electrical cable can also be transferred to the sensor, and insufficient isolation of electrical connections (e.g., with an electrically isolating material) can cause interruption of the signal through short circuits with the surrounding fluid.

These issues are addressed by the present disclosure through a rigid housing or casing (e.g., not a flexible tube) generally comprising one or more materials that can, for example, be laid down in layers to produce a 3D structure that isolates the sensor from stresses in the core wire, shaping ribbon, and/or other elements in the distal portion of the guidewire. In an example, the case or housing may be shaped approximately as a rounded rectangular prism, with 5 closed sides and one (partially) open side to expose the diaphragm. Alternatively, the case or housing may be approximately cylindrical and may comprise an approximately rectangular opening to accommodate the sensor. The case or housing may further include an integrated elevated platform to support the proximal side of the sensor, creating a cantilever. Microcables/filars may be bonded to the sensor (e.g., ultrasonically welded, wirebonded, eutectically bonded, or soldered).

The novel structures disclosed herein originate from the knowledge gained though projects for the development of pressure sensors for pressure-sensing guidewires and development of new pressure-sensing guidewires. Thus, the sensor casing or sensor housing of the present disclosure provides improved manufacturability by a reduction in processes now required to attach the core wire, conductive filars or conductors, and tubing to the sensor. The disclosed sensor casing or sensor housing also facilitates mounting of the sensor to the casing or housing at the end of the production line, which may tend to minimize sensor handling and thus reduce opportunities for sensor damage.

Some embodiments may include a diaphragm pillow, comprising a space around the sensing element of the sensor (e.g., a diaphragm) that is partially enclosed, but provides a gap between the housing and the cantilevered portion of the sensor. The size and shape of the gap are selected to trap air underneath the sensor and prevent it from escaping, thus creating a cantilever supported by air with minimum stress. In some embodiments, a soft material other than air may fill the gap to create the diaphragm pillow.

Some embodiments may include fluid flow ducts to provide fluid access to, and air escape from, the space under the cantilevered portion of the sensor. This may include for example a duct running parallel to the length of the sensor. This may be particularly important in embodiments where the diaphragm is facing inward, toward the core wire, to supply blood flow to the diaphragm location and prevent an air bubble from interfering with the operation of the diaphragm, which could potentially lead to incorrect sensor readings.

In some embodiments, the case or housing includes a bridge element above the diaphragm, with a space between the diaphragm and the bridge element for fluid access and air escape. The bridge element may serve to protect the diaphragm from contact with body surfaces (e.g., blood vessel side walls) during use, and/or from contact with other objects during assembly, storage, or handling of the guidewire device. In other embodiments, a bridge element spaced from the diaphragm may serve as a sensor clamp, providing a surface parallel to the bottom and top or sides of the sensor where the sensor can be slid between the two surfaces, with little or no gap between the sensor clamp and the sensor. In some embodiments, electrical traces may be integrated with the contact surfaces.

Some embodiments may include structures to provide support, protection, or routing for the conductive filars of the electrical microcable. For example, some embodiments provide electrical cable support parallel to the sensor electrical connection surface. This can be applicable for embodiments where the sensor is mounted with the diaphragm facing either up (away from the core wire) and/or down (toward the core wire).

Some embodiments may include adhesive flow channels or recesses to guide the flow of an adhesive used for mounting the sensor to the case or housing, to prevent adhesive flow towards the diaphragm, to limit the adhesive from flowing beyond a restricted outer diameter, and/or guide adhesive flow towards the underside of the sensor, thus encouraging continuous coverage of the adhesive in the areas where the underside of the sensor contacts the case or housing.

Example devices incorporating a multi-filar conductor bundle and/or conductive ribbons include intraluminal medical guidewire devices as described for example in U.S. Pat. No. 10,595,820 B2, U.S. Patent Publication Nos. 2014/0187874, 2016/0058977, and 2015/0273187, and in U.S. Provisional Patent Application No. 62/552,993 (filed Aug. 31, 2017), each of which is hereby incorporated by reference in its entirety as though fully set forth herein.

These descriptions are provided for exemplary purposes only, and should not be considered to limit the scope of the sensor housing or related assemblies. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Further, while the embodiments of the present disclosure may be described with respect to a blood vessel, it will be understood that the devices, systems, and methods described herein may be configured for use in any suitable anatomical structure or body lumen including a blood vessel, blood vessel lumen, an esophagus, eustachian tube, urethra, fallopian tube, intestine, colon, and/or any other suitable anatomical structure or body lumen. In other embodiments, the devices, systems, and methods described herein may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood vessels, chambers or other parts of the heart, and/or other systems of the body. In addition to natural structures, the device 102 may be used to examine man-made structures such as, but without limitation, heart valves, stents, shunts, filters and other devices. For the sake of brevity, however, the numerous iterations of these combinations will not be described separately.

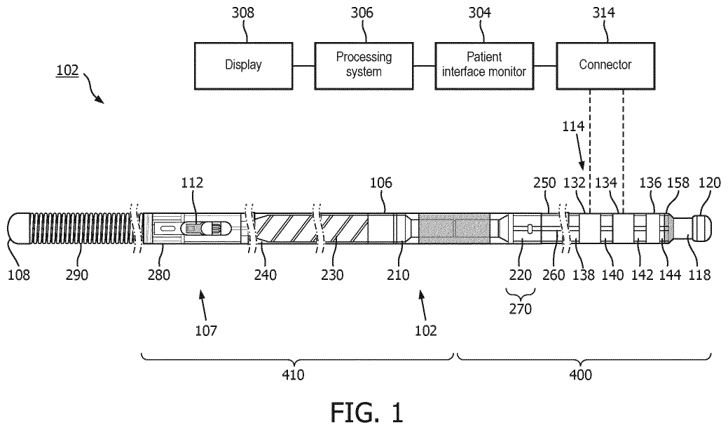

FIG. 1 is a diagrammatic side view of an intraluminal (e.g., intravascular) sensing system 100 that includes an intravascular device 102 comprising conductive members 230 (e.g., a multi-filar electrical conductor bundle) and conductive ribbons 260, according to aspects of the present disclosure. The intravascular device 102 can be an intravascular guidewire sized and shaped for positioning within a vessel of a patient. The intravascular device 102 includes a distal tip 108 and an electronic component 112. For example, the electronic component 112 can be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor can be used to calculate physiological variables such as coronary flow reserve (CFR). The intravascular device 102 includes a flexible elongate member 106. The electronic component 112 is disposed at a distal portion 107 of the flexible elongate member 106. The electronic component 112 can be mounted at the distal portion 107 within a housing 280 in some embodiments. A flexible tip coil 290 extends distally from the housing 280 at the distal portion 107 of the flexible elongate member 106. A connection portion 114 located at a proximal end of the flexible elongate member 106 includes conductive portions 132, 134. In some embodiments, the conductive portions 132, 134 can be conductive ink that is printed and/or deposited around the connection portion 114 of the flexible elongate member 106. In some embodiments, the conductive portions 132, 134 are conductive, metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 1 includes core wire comprising a distal core 210 and a proximal core 220. The distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 can be made of a metal or metal alloy. For example, the distal core 210 and/or the proximal core 220 can be made of stainless steel, Nitinol, nickel-cobalt-chromium-molybdenum alloy (e.g., MP35N), and/or other suitable materials. In some embodiments, the distal core 210 and the proximal core 220 are made of the same material. In other embodiments, the distal core 210 and the proximal core 220 are made of different materials. The diameter of the distal core 210 and the proximal core 220 can vary along their respective lengths. A joint between the distal core 210 and proximal core 220 is surrounded and contained by a hypotube 215. The electronic component 112 can in some cases be positioned at a distal end of the distal core 210.

In some embodiments, the intravascular device 102 comprises a distal subassembly and a proximal subassembly that are electrically and mechanically joined together, which creates an electrical communication between the electronic component 112 and the conductive portions 132, 134. For example, flow data obtained by the electronic component 112 (in this example, electronic component 112 is a flow sensor) can be transmitted to the conductive portions 132, 134. In an exemplary embodiment, the flow sensor 112 is a single ultrasound transducer element. In some embodiments, the transducer element emits ultrasound signals, receives echoes, and generates electrical signals representative of the echoes. The processing system 306 processes the electrical signals to extract the flow velocity of the fluid. In some embodiments, the electronic component is a pressure transducer (e.g., based on piezoresistive technology) and generates electrical signals representative of the pressure within the vessel. The signal carrying filars carry these electrical signals from the sensor at the distal portion to the connector at the proximal portion.

Control signals from a processing system 306 (e.g., a processor circuit of the processing system 306) in communication with the intravascular device 102 can be transmitted to the electronic component 112 via a connector 314 that attached to the conductive portions 132, 134. The distal subassembly can include the distal core 210. The distal subassembly can also include the electronic component 112, the conductive members 230, and/or one or more layers of insulative polymer/plastic 240 surrounding the conductive members 230 and the core 210. For example, the polymer/plastic layer(s) can insulate and protect the conductive members of the multi-filar cable or conductor bundle 230. The proximal subassembly can include the proximal core 220. The proximal subassembly can also include one or more polymer layers 250 (hereinafter polymer layer 250) surrounding the proximal core 220 and/or conductive ribbons 260 embedded within the one or more insulative and/or protective polymer layer 250. In some embodiments, the proximal subassembly and the distal subassembly are separately manufactured. During the assembly process for the intravascular device 102, the proximal subassembly and the distal subassembly can be electrically and mechanically joined together. As used herein, flexible elongate member can refer to one or more components along the entire length of the intravascular device 102, one or more components of the proximal subassembly (e.g., including the proximal core 220, etc.), and/or one or more components the distal subassembly 410 (e.g., including the distal core 210, etc.). Accordingly, flexible elongate member may refer to the combined proximal and distal subassemblies described above. The joint between the proximal core 220 and distal core 210 is surrounded by the hypotube 215.

In various embodiments, the intravascular device 102 can include one, two, three, or more core wires extending along its length. For example, a single core wire can extend substantially along the entire length of the flexible elongate member 106. In such embodiments, a locking section 118 and a section 120 can be integrally formed at the proximal portion of the single core wire. The electronic component 112 can be secured at the distal portion of the single core wire. In other embodiments, such as the embodiment illustrated in FIG. 1, the locking section 118 and the section 120 can be integrally formed at the proximal portion of the proximal core 220. The electronic component 112 can be secured at the distal portion of the distal core 210. The intravascular device 102 includes one or more conductive members 230 (e.g., a multi-filar conductor bundle or cable) in communication with the electronic component 112. For example, the conductive members 230 can be one or more electrical wires that are directly in communication with the electronic component 112. In some instances, the conductive members 230 are electrically and mechanically coupled to the electronic component 112 by, e.g., soldering. In some instances, the conductor bundle 230 comprises two or three electrical wires (e.g., a bifilar cable or a trifilar cable). An individual electrical wire can include a bare metallic conductor surrounded by one or more insulating layers. The conductive members 230 can extend along the length of the distal core 210. For example, at least a portion of the conductive members 230 can be spirally wrapped around the distal core 210, minimizing or eliminating whipping of the distal core within tortuous anatomy.

The intravascular device 102 includes one or more conductive ribbons 260 at the proximal portion of the flexible elongate member 106. The conductive ribbons 260 are embedded within polymer layer 250. The conductive ribbons 260 are directly in communication with the conductive portions 132 and/or 134. In some instances, a multi-filar conductor bundle 230 is electrically and mechanically coupled to the electronic component 112 by, e.g., soldering. In some instances, the conductive portions 132 and/or 134 comprise conductive ink (e.g., metallic nano-ink, such as copper, silver, gold, or aluminum nano-ink) that is deposited or printed directed over the conductive ribbons 260.

As described herein, electrical communication between the conductive members 230 and the conductive ribbons 260 can be established at the connection portion 114 of the flexible elongate member 106. By establishing electrical communication between the conductor bundle 230 and the conductive ribbons 260, the conductive portions 132, 134 can be in electrical communication with the electronic component 112.

In some embodiments represented by FIG. 1, the intravascular device 102 includes a locking section 118 and a retention section 120. To form locking section 118, a machining process is used to remove polymer layer 250 and conductive ribbons 260 in locking section 118 and to shape proximal core 220 in locking section 118 to the desired shape. As shown in FIG. 1, locking section 118 includes a reduced diameter while retention section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 260.

In some embodiments, a connector 314 provides electrical connectivity between the conductive portions 132, 134 and a patient interface monitor 304. The Patient Interface Monitor 304 may in some cases connect to a console or processing system 306, which includes or is in communication with a display 308.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some embodiments, device 102 may be controlled from a remote location such as the control room, such that an operator is not required to be in close proximity to the patient.

The intraluminal device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other embodiments, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 can include a processor and a memory. The processing system 306 may be operable to facilitate the features of the intravascular sensing system 100 described herein. For example, the processor can execute computer readable instructions stored on the non-transitory tangible computer readable medium.

The PIM 304 facilitates communication of signals between the processing system 306 and the intraluminal device 102. The PIM 304 can be communicatively positioned between the processing system 306 and the intraluminal device 102. In some embodiments, the PIM 304 performs preliminary processing of data prior to relaying the data to the processing system 306. In examples of such embodiments, the PIM 304 performs amplification, filtering, and/or aggregating of the data. In an embodiment, the PIM 304 also supplies high-and low-voltage DC power to support operation of the intraluminal device 102 via the conductive members 230.

A multi-filar cable or transmission line bundle 230 can include a plurality of conductors, including one, two, three, four, five, six, seven, or more conductors. In the example shown in FIG. 1, the multi-filar conductor bundle 230 includes two straight portions 232 and 236, where the multi-filar conductor bundle 230 lies parallel to a longitudinal axis of the flexible elongate member 106, and a spiral portion 234, where the multi-filar conductor bundle 230 is wrapped around the exterior of the flexible elongate member 106 and then overcoated with an insulative and/or protective polymer 240. Communication, if any, along the multi-filar conductor bundle 230 may be through numerous methods or protocols, including serial, parallel, and otherwise, wherein one or more filars of the bundle 230 carry signals. One or more filars of the multi-filar conductor bundle 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some embodiments, the display 308 may be used to provide a procedure-specific workflow to a user to complete an intraluminal imaging procedure.

Before continuing, it should be noted that the examples described above are provided for purposes of illustration, and are not intended to be limiting. Other devices and/or device configurations may be utilized to carry out the operations described herein.

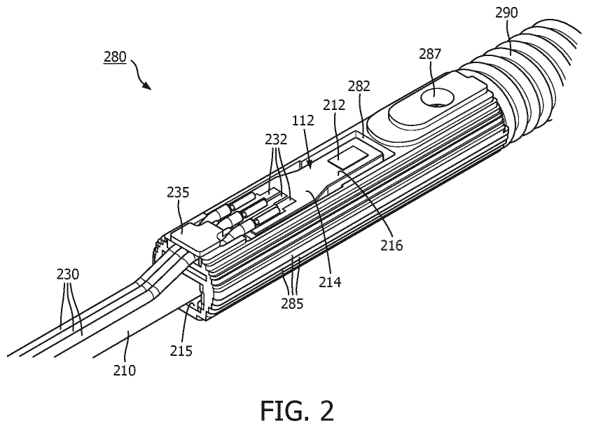

FIG. 2 is a diagrammatic, perspective view of at least a portion of an example sensor housing 280, in accordance with at least one embodiment of the present disclosure. In the example shown in FIG. 2, the sensor housing 280 is built up from a plurality of layers 285, and includes a core wire lumen 215, conductor support 235, sensor cavity 282 and optional attachment hole 287. The core wire lumen 215 is configured to receive the distal core wire 210. The conductor support is positioned near a distal end of the sensor housing 280, and is configured to guide the conductors or filars 230 toward the weld pads 232 on the sensor 112. In an example, the filars 230 are electrically bonded (e.g., by solder, conductive adhesive, ultrasonic welding, or other means) to the weld pads 232. The conductor support 235 may at least partially protect the filars 230, and/or the connection between the filars 230 and the weld pads 232, from mechanical stress, and may also reduce the transfer to the sensing element 212 of mechanical stresses on the filars 230. The weld pads 232 of the sensor 112 are located on a fixed portion 214 of the sensor 112, a diaphragm or other sensing element 212 is located on a cantilevered portion 216 of the sensor 112, which is disposed within the sensor cavity 282. A flexible tip coil 290 extends distally from the sensor housing 280. The sensor cavity 282 may, in some embodiments, be in fluid communication with at least a portion of the core wire lumen 215. For example, there could be one or more holes between the sensor cavity 282 and the core wire lumen 215 for the fluid communication. The core wire lumen and the open sensor cavity can be different even with fluid communication because the structure of the sensor housing defines different areas (core wire lumen and sensor cavity). In some embodiments, the bottom surface of the open sensor cavity 282 may be deleted altogether, such that the bottom of the sensor cavity 282 opens into the core wire lumen 215.

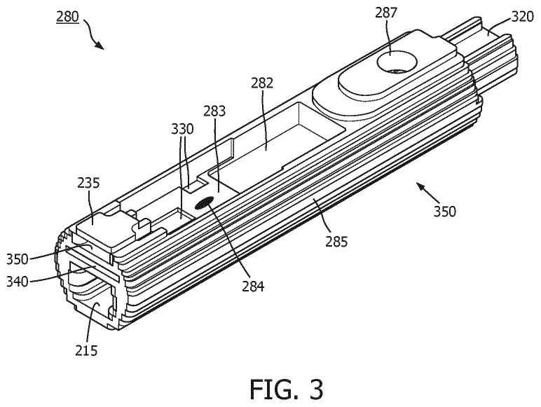

FIG. 3 is a diagrammatic, proximal end view of at least a portion of an example sensor housing 280, in accordance with at least one embodiment of the present disclosure. In the example shown in FIG. 3, the sensor housing 280 includes a sensor cavity 282, within which a sensor attachment platform 283 provides support for the fixed portion 214 of the sensor 112 (see FIG. 2). The sensor cavity 282 is configured to receive the cantilevered portion 216 of the sensor 112 (see FIG. 2), such that the bottom surface of the cantilevered portion 216 is not in contact with the bottom surface of the sensor cavity 282. This cantilevered configuration may tend to isolate the sensor diaphragm 212 (see FIG. 2) from stresses and vibration. Visible are the plurality of layers 285, core wire lumen 215, attachment hole 287, a conductor opening 350 situated beneath the conductor support 235, and a shaping ribbon channel 320 for receiving a shaping ribbon.

In most embodiments, the sensor housing 280 is formed from substantially rigid materials such as metal or rigid polymer. However, it is recognized that many materials that would be rigid at larger sizes may exhibit at least some degree of flexibility in thicknesses smaller than one millimeter. Although the sensor housing 280 comprises a plurality of layers 285, these may for example be layers formed by a 3D printing process or semiconductor fabrication process, as disclosed for example in U.S. patent application Ser. No. 17/188,012 to Burkett et al., filed Mar. 1, 2021, hereby incorporated by reference as though fully set forth herein. Thus, even where two or more different materials are incorporated into the plurality of layers 285, the sensor housing 280 may be considered a single solid object/component (e.g., a monolithic object/component) rather than an assembly of multiple components. In some embodiments, the plurality of layers 285 form a generally cylindrical outer profile 350, such that the sensor housing 280 can form the outer profile of the guidewire at the location of the sensor housing 280. The core wire lumen 215 similarly forms a cylindrical shape, such that its cross section can completely surround a cross section of the core wire in at least some locations along the length of the sensor housing 280. For example, the same component (the sensor housing 280) forms the outer profile of the guidewire, defines a lumen for the guidewire, and defines the cavity in which the sensor is positioned. This advantageously eliminates the need to have two separate components (one component defining the lumen for the guidewire and defining a space where the sensor is positioned, and a separate component defining the outer profile of the guidewire), which have to be coupled to one another.

In the example shown in FIG. 3, the sensor cavity 282 includes adhesive flow channels or recesses 330 and an adhesive escape lumen 340, which are configured to receive and/or guide the flow of an adhesive 284 used for mounting the sensor 112 to sensor attachment platform 283. Such guiding or receiving may help prevent the adhesive 284 from flowing toward the diaphragm 212 or filling the space beneath the cantilevered portion 216. The channels or recesses 330 and adhesive escape lumen 340 may also help to limit the adhesive 284 from flowing beyond a restricted outer diameter, and/or may guide the flow of the adhesive towards the underside of the sensor 112, thus encouraging a desirable coverage between the sensor attachment platform 283 and the fixed portion 214 of the sensor 112.

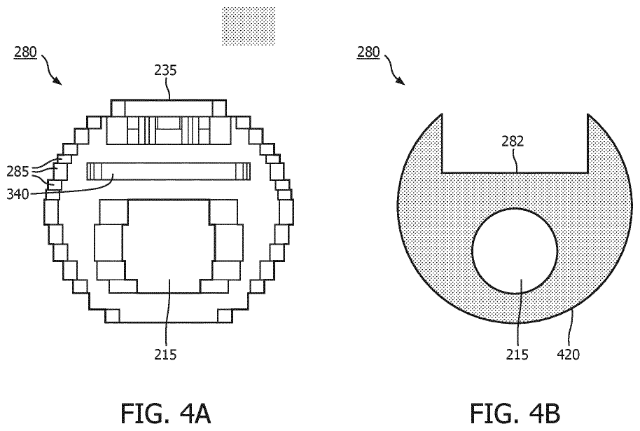

FIG. 4A is a diagrammatic, proximal end view of at least a portion of an example sensor housing 280, in accordance with at least one embodiment of the present disclosure. In the example shown in FIG. 4A, the sensor housing 280 comprises a plurality of stacked layers 285, each of which is shaped such that the plurality of layers 285 forms the features of the sensor housing 280 shown in FIGS. 2 and 3. These layers 285 may for example be laid down via a 3D printing process, or via deposition, masking, and etching processes similar to those used in semiconductor manufacturing or microelectomechanical systems (MEMS), or by other means. In some embodiments, the layers 285 comprise a conductive material such as a metal, a doped semiconductor, a doped ceramic, or a doped polymer. In other embodiments, the layers 285 comprise an insulative material such as a polymer, ceramic, or composite. In still other embodiments, one or more layers may comprise two or more different materials in order to provide electrical conduction pathways and/or electrical insulation, as described below.

Also visible are the core wire lumen 215, adhesive escape lumen 340, and conductor support 235.

FIG. 4B is a diagrammatic, proximal end view of at least a portion of an example sensor housing 280, in accordance with at least one embodiment of the present disclosure. In the example shown in FIG. 4B, the sensor housing 280 comprises a smooth shape 420, that may for example be formed by casting or machining, or by sintering or polishing of a layered structure such as the one depicted in FIG. 4A. Also visible are the core wire lumen 215 and sensor cavity 282. Depending on the implementation, the smooth continuous shape 420 may comprise any or all of the features disclosed herein for the sensor housing 280.

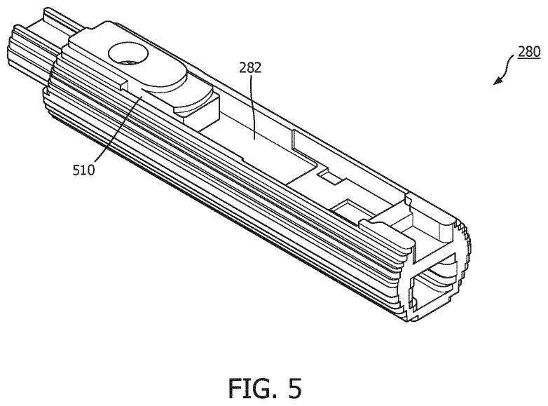

FIG. 5 is a diagrammatic, perspective view of at least a portion of an example sensor housing 280, in accordance with at least one embodiment of the present disclosure. In the example shown in FIG. 5, the sensor housing 280 includes fluid flow channels 510 that provide fluid communication between the sensor cavity 282 and the fluid (e.g., blood) surrounding the intravascular device 102 (see FIG. 1). The fluid flow channels 510 may for example provide fluid access to, and air escape from, the space under the cantilevered portion of the sensor. This may be particularly important in embodiments where the sensing element (e.g., a pressure-sensing diaphragm) is facing inward, toward the core wire, as the fluid flow channels 510 can supply blood flow to the sensing element, and thus prevent an air bubble from interfering with the operation of the sensing element, which could potentially lead to incorrect sensor readings.

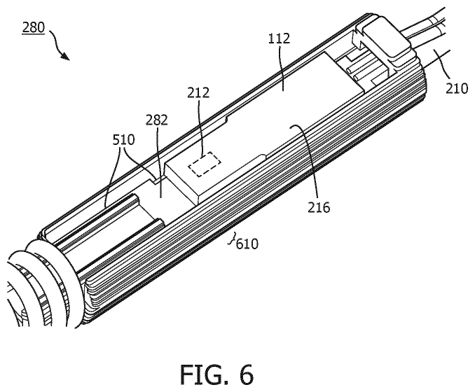

FIG. 6 is a diagrammatic, perspective view of at least a portion of an example sensor housing 280 that incorporates fluid flow channels 510, in accordance with at least one embodiment of the present disclosure. In the example shown in FIG. 6, the sensor 112 has been placed in the sensor cavity 282 in a face-down configuration, such that the diaphragm or sensing element 212 faces downward into the sensor cavity 282 (e.g., radially toward the distal core wire 210) rather than facing upward (e.g., radially away from the distal core wire 210) as shown in FIG. 2. This downward-facing configuration may for example provide protection to the diaphragm or sensing element 212, preventing objects or surfaces from contacting the diaphragm or sensing element 212 during assembly, storage, handling, or use of the intravascular device.

However, in this configuration, any air that becomes trapped below the cantilevered portion 216 of the sensor 112 can potentially interfere with the sensing element 212 and thus affect sensor readings. Thus, the fluid flow channels 510 are configured to provide a pathway for air to escape from, and fluid (e.g., blood) to enter, a gap 610 that exists between the bottom surface of the cantilevered portion 216 and the bottom surface of the sensor cavity 282, such that the sensing element 212 is surrounded by the fluid rather than by air.

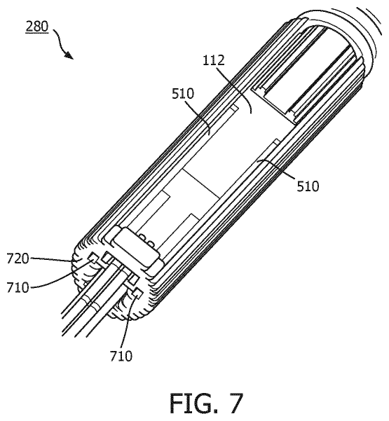

FIG. 7 is a diagrammatic, perspective view of at least a portion of an example sensor housing 280 that incorporates fluid flow channels 510, in accordance with at least one embodiment of the present disclosure. In the example shown in FIG. 7, the sensor 112 has been placed in the sensor cavity 282 in a face-down configuration, and the fluid flow channels 510 are configured to provide a pathway for air to escape from, and fluid (e.g., blood) to enter, a gap between the bottom surface of the sensor 112 and the bottom surface of the sensor cavity 282, such that the sensing element can be surrounded by fluid rather than by air. In some embodiments, the fluid flow channels 510 may be in fluid communication with fluid flow lumens 710 which exit through the proximal face 720 of the sensor housing 280.

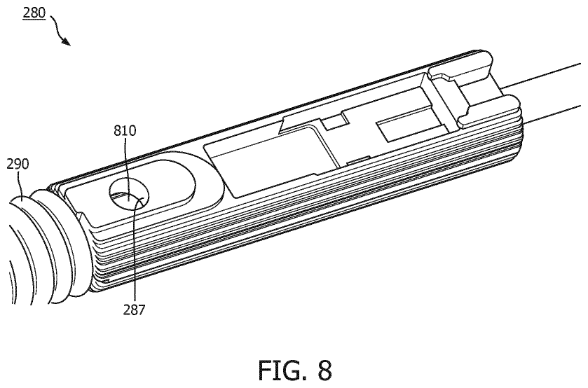

FIG. 8 is a diagrammatic, perspective view of at least a portion of an example sensor housing 280, in accordance with at least one embodiment of the present disclosure. In the example shown in FIG. 8, a portion of a shaping ribbon 810 is visible below the attachment hole 287. In an example, the shaping ribbon 810 may extend from partially within the sensor housing 280 to partially within the flexible tip coil 290. Thus, an application of solder or adhesive to the attachment hole 287 may serve to bond at least a portion of the shaping ribbon 810 to the sensor housing 280. Other embodiments may not include an attachment hole 287.

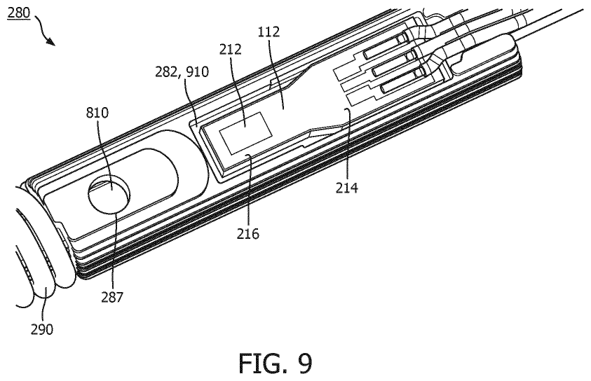

FIG. 9 is a diagrammatic, perspective view of at least a portion of an example sensor housing 280, in accordance with at least one embodiment of the present disclosure. In the example shown in FIG. 9, a diaphragm pillow 910 is formed within the sensor cavity 282, to cushion potential stress or vibration of the cantilevered portion 216, and thus help to mechanically isolate the diaphragm or sensing element 212. Depending on the implementation, the diaphragm pillow may comprise a soft material such as foam, gel, or soft adhesive, or may comprise air that is trapped beneath the cantilevered portion 216. In embodiments that include a diaphragm pillow 910 comprising trapped air, it may be undesirable for the sensor housing 280 to include fluid flow channels that might permit the trapped air to escape.

Also visible are the fixed portion 214 of the sensor 212, along with the attachment hole 287, shaping ribbon 810, and flexible tip coil 290.

Some embodiments may include a diaphragm pillow, comprising a space around the sensing element of the sensor (e.g., a diaphragm) that is partially enclosed, but provides a gap between the housing and the cantilevered portion of the sensor. The size and shape of the gap are selected to trap air underneath the sensor and prevent it from escaping, thus creating a cantilever supported by air with minimum stress. In some embodiments, a soft material other than air may fill the gap to create the diaphragm pillow.

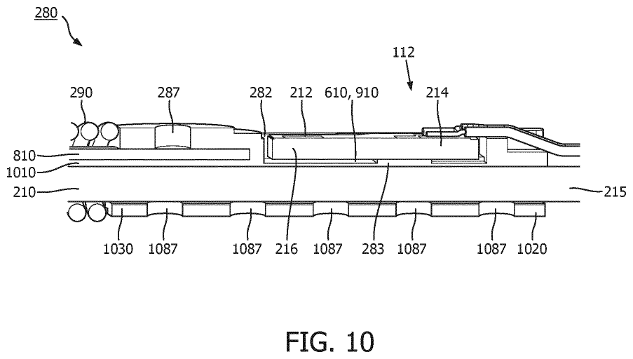

FIG. 10 is a diagrammatic, cross-sectional view of at least a portion of an example sensor housing 280, in accordance with at least one embodiment of the present disclosure. In the example shown in FIG. 10, the sensor 112, sensor cavity 282, and sensor attachment platform 283 are configured such that a gap 610 is formed between the bottom of the cantilevered portion 216 and the bottom of the sensor cavity 282. In some embodiments, the sensor 112, sensor cavity 282, sensor attachment platform 283, and gap 610 are configured such that air can become trapped within the gap 610, forming a diaphragm pillow 910 that may help to isolate the diaphragm or sensing element 212 from stress or vibration elsewhere within the intravascular device.

Also visible is the shaping ribbon 810, which extends from a distal portion of the sensor housing 280 through at least a proximal portion of the flexible tip coil 290. The flexible tip coil 290 may for example be attached to a distal connection feature 1010 of the sensor housing 280 by means of solder or adhesive. Similarly, attachment hole 287 extends through a top surface of the sensor housing 280 to the shaping ribbon 810, and may be used to inject or apply solder or adhesive, forming a bond between the shaping ribbon 810 and the sensor housing 280. In other embodiments, the sensor housing 280 may be bonded to the shaping ribbon 810 by ultrasonic welding, or other means.

The distal core wire 210 extends through the core wire lumen 215, extending proximal of the proximal end 1020 and distal of the distal end 1030 of the sensor housing 280. Attachment holes 1087 extend through a bottom surface of the sensor housing 280 to the distal core wire 210, and may be used to inject or apply solder or adhesive, forming a bond between the distal core wire 210 and the sensor housing 280. In some embodiments, the sensor housing 280 may be bonded to the distal core wire 210 by ultrasonic welding, or other means.

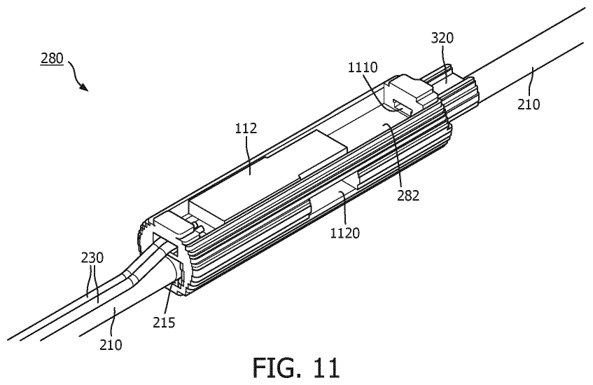

FIG. 11 is a diagrammatic, perspective view of at least a portion of an example sensor housing 280, in accordance with at least one embodiment of the present disclosure. In the example shown in FIG. 11, the sensor housing 280 includes a receiving channel 320 for the shaping ribbon, a shaping ribbon lumen 1110, and a fluid flow lumen 1120 in the side of the sensor housing 280 that may for example be in fluid communication, whether directly or indirectly, with the sensor cavity 282 generally, or with the gap 610 (see FIG. 6) in particular. In some embodiments, one or more fluid flow lumens 1120 may be located in the top, bottom, sides, distal end, or proximal end of the sensor housing 280. Also visible are the distal core wire 210, conductive filars 230, and sensor 112.

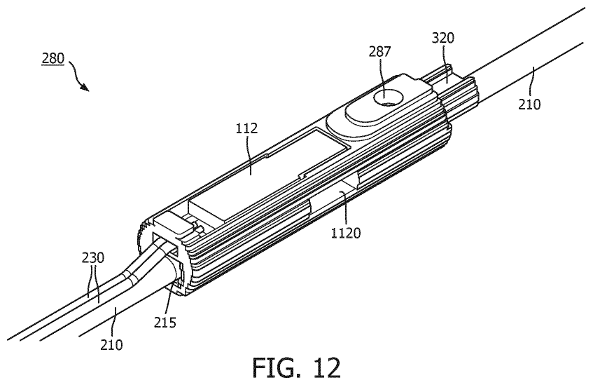

FIG. 12 is a diagrammatic, perspective view of at least a portion of an example sensor housing 280, in accordance with at least one embodiment of the present disclosure. The example sensor housing 280 shown in FIG. 12 is similar to that of FIG. 11, except that it includes an attachment hole 287 rather than a shaping ribbon lumen 1110.

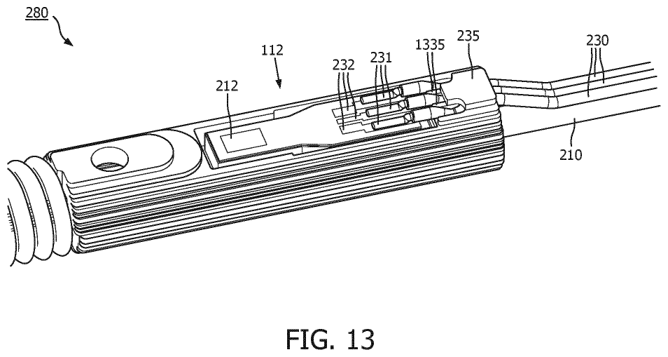

FIG. 13 is a diagrammatic, perspective view of at least a portion of an example sensor housing 280, in accordance with at least one embodiment of the present disclosure. In the example shown in FIG. 13, the sensor 112 is controlled by the filars 230, which are bonded to respective weld pads 232. The conductors are held in place by a conductor support 235, which includes two spreading tines 1335. Because the filars 230 are grouped into a single cable, in some embodiments, the filars 230 may not be bonded to the weld pads 232 unless they are physically separated from one another and spread out slightly, such that the pitch or spacing of the distal filar ends 231 matches that of the weld pads 232. Even under high magnification, this separation and spreading may be difficult to perform manually, and may also be difficult to automate. The spreading tines 1335 of the conductor support 235 may be configured to assist in the separation and spreading of the filars 230, and positioning of the filar ends 231 for bonding to the weld pads 232. Two spreading tines 1335 are shown, for spreading three filars 230. However, other configurations are possible, including but not limited to one spreading tine 1335 to separate two filars 230, three spreading tines to separate four filars 230, etc.

It should be noted that in some embodiments, a conductive core wire 210 may be used to carry electrical current and/or signals, and may thus eliminate the need for one of the filars 230, so that (for example) a trifilar cable may be replaced with a bifilar cable plus conductive core wire. In such embodiments, the sensor 112 may be configured with one electrical lead or bond pad that is configured to connect directly to a conductive sensor housing 280. Thus, the sensor housing 280 may serve as an electrical connection, as well as a physical housing, for the sensor 112, such that the sensor 112, when installed in the sensor housing 280, is in electrical communication with the core wire 210.

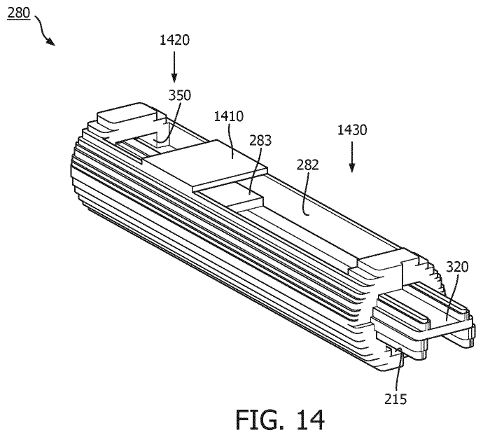

FIG. 14 is a diagrammatic, perspective view of at least a portion of an example sensor housing 280, in accordance with at least one embodiment of the present disclosure. Visible are the sensor cavity 282, shaping ribbon receiving channel 320, and conductor opening 350. In the example shown in FIG. 14, the sensor housing 280 includes a bridge element 1410, extending laterally across the sensor cavity 282 near a proximal end 1420 of the sensor cavity 282, above or approximately above the attachment platform 283. In some embodiments, a bridge element 1410 in this or a similar position may serve as a sensor clamp, configured to apply downward pressure to the sensor 112 (see FIG. 2) and thus help to hold the sensor 112 in place.

In other embodiments, a bridge element 1410 positioned across a more central region 1430 of the sensor cavity 282 may serve to protect the sensing element 212 of the sensor 112 (see FIG. 2) from direct contact during use or handling of the intravascular device. In such embodiments, the bridge element 1410 may be arched or otherwise configured to provide clearance between the bottom surface of the bridge element 1410 and the top surface of the sensing element 212, both to prevent contact between the bridge element 1410 and the sensing element 212 and to permit fluid flow between the bridge element 1410 and the sensing element 212.

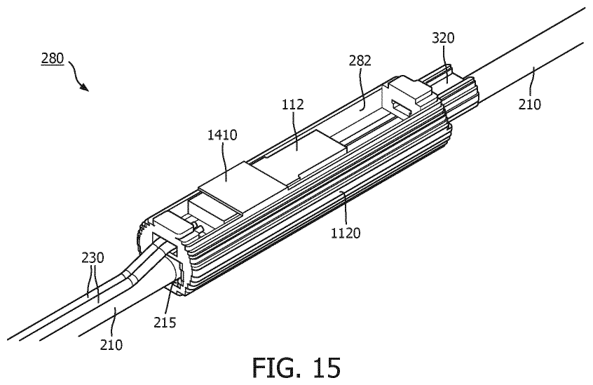

FIG. 15 is a diagrammatic, perspective view of at least a portion of an example sensor housing 280, in accordance with at least one embodiment of the present disclosure. Visible are the distal core wire 210, core wire lumen 215, filars 230, and shaping ribbon receiving channel 320. In the example shown in FIG. 15, the sensor housing 280 includes a bridge element 1410, extending laterally across the sensor cavity 282 near a proximal of the sensor cavity 282. The bridge element 1410 serves as a sensor clamp, configured to apply downward pressure to the sensor 112 and thus help to hold the sensor 112 in place. In some embodiments, the bridge element/sensor clamp 1410 may be configured to help isolate the sensor 112 from vibration and mechanical stresses occurring elsewhere in the intravascular device. For example, a stress-and vibration-absorbing flexible adhesive may be positioned between the bottom surface of the bridge element 1410 and the top surface of the sensor.

In some embodiments, one or more isolated electrical traces may be integrated with the bottom surface of the bridge element, in positions aligned with the weld pads 232 of the sensor 112 (see FIG. 2), to form electrical connections between the sensor 112 and the filars 230 and/or core wire 215.

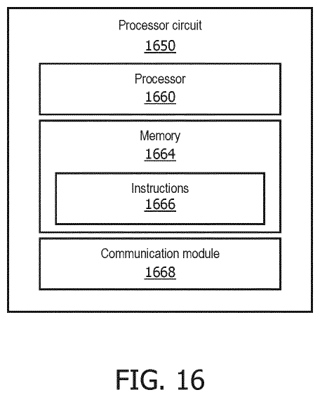

FIG. 16 is a schematic diagram of a processor circuit 1650, according to at least one embodiment of the present disclosure. The processor circuit 1650 may be implemented in the intravascular sensing system 100 (e.g., the PIM 304, processing system 306) or other devices or workstations (e.g., third-party workstations, network routers, etc.), or on a cloud processor or other remote processing unit, as necessary to implement the method. As shown, the processor circuit 1650 may include a processor 1660, a memory 1664, and a communication module 1668. These elements may be in direct or indirect communication with each other, for example via one or more buses.

The processor 1660 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1660 may also comprise another hardware device, a firmware device, or any combination thereof configured to perform the operations described herein. The processor 1660 may also be implemented as a combination of computing devices, e.g., a combination of a DSP and a microprocessor, a plurality of microprocessors, one or more microprocessors in conjunction with a DSP core, or any other such configuration.

The memory 1664 may include a cache memory (e.g., a cache memory of the processor 1660), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1664 includes a non-transitory computer-readable medium. The memory 1664 may store instructions 1666. The instructions 1666 may include instructions that, when executed by the processor 1660, cause the processor 1660 to perform the operations described herein. Instructions 1666 may also be referred to as code. The terms “instructions” and “code” should be interpreted broadly to include any type of computer- readable statement(s). For example, the terms “instructions” and “code” may refer to one or more programs, routines, sub-routines, functions, procedures, etc. “Instructions” and “code” may include a single computer-readable statement or many computer-readable statements.

The communication module 1668 can include any electronic circuitry and/or logic circuitry to facilitate direct or indirect communication of data between the processor circuit 1650, and other processors or devices. In that regard, the communication module 1668 can be an input/output (I/O) device. In some instances, the communication module 1668 facilitates direct or indirect communication between various elements of the processor circuit 1650 and/or the intravascular measurement system 100. The communication module 1668 may communicate within the processor circuit 1650 through numerous methods or protocols. Serial communication protocols may include but are not limited to US SPI, I²C, RS-232, RS-485, CAN, Ethernet, ARINC 429, MODBUS, MIL-STD-1553, or any other suitable method or protocol. Parallel protocols include but are not limited to ISA, ATA, SCSI, PCI, IEEE-488, IEEE-1284, and other suitable protocols. Where appropriate, serial and parallel communications may be bridged by a UART, USART, or another appropriate subsystem.

External communication (including but not limited to software updates, firmware updates, preset sharing between the processor and central server, or readings from the intraluminal device) may be accomplished using any suitable wireless or wired communication technology, such as a cable interface such as a USB, micro USB, Lightning, or FireWire interface, Bluetooth, Wi-Fi, ZigBee, Li-Fi, or cellular data connections such as 2G/GSM, 3G/UMTS, 4G/LTE/WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

What is claimed is:

1. An intraluminal sensing device, comprising:
a guidewire configured to be positioned within a body lumen of a patient, wherein the guidewire comprises:
a core wire;
a sensor configured to obtain medical data associated with the body lumen; and
a sensor housing fixedly attached to the core wire, wherein the sensor housing is a single component and defines an outer profile of the guidewire, wherein the sensor housing comprises:
a core wire lumen configured to receive the core wire through an entire length of the sensor housing such that a cross section of the core wire lumen completely surrounds a cross section of the core wire; and
an open sensor cavity different from the core wire lumen and configured to receive the sensor,
wherein the sensor cavity comprises an attachment platform fixedly attached to a fixed portion of the sensor such that a cantilevered portion of the sensor is suspended within the sensor cavity.

2. The intraluminal sensing device of claim 1, wherein the guidewire further comprises a pillow disposed beneath the cantilevered portion of the sensor.

3. The intraluminal sensing device of claim 2, wherein the pillow comprises trapped air.

4. The intraluminal sensing device of claim 1, wherein the sensor housing further comprises an adhesive flow channel or an adhesive flow recess configured to receive an excess of an adhesive, wherein the attachment platform is fixedly attached to the fixed portion of the sensor by the adhesive.

5. The intraluminal sensing device of claim 1, wherein the sensor housing further comprises a fluid flow channel configured to permit access to a sensing element of the sensor by a fluid within the body lumen of the patient.

6. The intraluminal sensing device of claim 1, wherein the sensor housing further comprises at least one core wire solder hole configured to receive solder or adhesive, such that the core wire is fixedly attached to the sensor housing by the solder or adhesive.

7. The intraluminal sensing device of claim 1, wherein the guidewire further comprises a shaping ribbon extending distally from the sensor housing into a flexible tip coil, wherein the flexible tip coil is fixedly attached to the sensor housing at a distal end of the sensor housing.

8. The intraluminal sensing device of claim 7, wherein the sensor housing further comprises:
a channel for receiving the shaping ribbon; or
at least one shaping ribbon attachment hole configured to receive solder or adhesive, such that the shaping ribbon is fixedly attached to the sensor housing by the solder or adhesive.

9. The intraluminal sensing device of claim 1,
wherein the guidewire further comprises at least one conductor in electrical communication with the sensor,
wherein the sensor housing further comprises a conductor support configured to reduce a transference of mechanical stress from the at least one conductor to the sensor.

10. The intraluminal sensing device of claim 9, wherein the at least one conductor comprises at least two conductors, and wherein the conductor support further comprises a separation tine for separating two conductors of the at least two conductors.

11. The intraluminal sensing device of claim 1, wherein the sensor cavity is configured to receive the sensor such that a sensing element of the sensor, positioned on the cantilevered portion of the sensor, is facing radially inward toward the core wire, such that a gap exists between the sensing element and a bottom surface of the sensor cavity.

12. The intraluminal sensing device of claim 1, wherein the sensor cavity is configured to receive the sensor such that a sensing element of the sensor is facing radially outward, away from the core wire.

13. The intraluminal sensing device of claim 1, wherein the sensor housing further comprises a bridge element extending laterally across the sensor cavity, such that a bottom surface of the bridge element is not in contact with a bottom surface of the sensor cavity.

14. The intraluminal sensing device of claim 13, wherein the bottom surface of the bridge element is configured to apply a downward pressure to a top surface of the fixed portion of the sensor.

15. The intraluminal sensing device of claim 13, wherein the bridge element is configured to provide a space between the bottom surface of the bridge element and a top surface of the cantilevered portion of the sensor.
